(19) United States
(12) Patent Application Publication
(10) Pub. No.: US 2009/0209600 A1
(43) Pub. Date: Aug. 20, 2009

(54) MOISTURIZATION IMPROVEMENT VIA A HEATED NIACINAMIDE CONTAINING FLOWABLE COSMETIC TRANSFERRED ONTO SKIN

(75) Inventors: Philip Edward Miner, Newtown, CT (US); Stephen Roy Barrow, Trumbull, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/361,604

(22) Filed: Jan. 29, 2009

Related U.S. Application Data
(60) Provisional application No. 61/029,403, filed on Feb. 18, 2008.

(51) Int. Cl. A61K 31/44

(52) U.S. Cl. 514/356

(57) ABSTRACT

A product and method for moisturizing skin is herein described. The product is a package with associated instructions for applying a niacinamide containing cosmetic composition to skin dispensed from a heating device. The heated cosmetic composition imparts a more rapid moisturization than expected from an identical composition applied at room temperature.

### BACKGROUND OF THE INVENTION

1. Field of the Invention

The invention concerns a product for improving moisturization that includes a niacinamide formulated flowable cosmetic composition dispensed from a heating device.

2. The Related Art

Dry skin is a problem in varying degree to most humans. This condition is particularly evident in winter. Personal care products such as skin creams/lotions, shampoos/conditioners, toilette bars/shower gels and antiperspirant/deodorants are normally formulated with at least one material to address dry skin. Symptoms such as itching, flaking and a visually displeasing dermal appearance can all to some extent be modulated.

There are three classes of materials employed against the problem. Occlusives such as petrolatum or silicone oils serve to inhibit loss of natural moisture. They form a barrier between the epidermis and the environment. Another approach is the use of keratolytic agents to enhance rate of dermal exfoliation. Alpha-hydroxy acids are the most common agents for achieving exfoliation.

A third approach to dry skin is topical application of humectants. Hydroxylated monomeric and polymeric organic substances are generally used for this purpose. Glycerin known also as glycerol is one of the most effective humectants.

There are several shortcomings in the performance of known humectants. Even the best such as glycerin requires to be formulated at relatively high levels to achieve good moisturization. Secondly, known humectants perform well in high relative humidity environments; however, hardly any of these substances provide effectiveness at low relative humidity (i.e. less than 20% moisture at 20° C.). Average indoor relative humidity during winter is approximately 13% in areas such as the Northeast U.S. It is quite evident that a real need exists for an improved moisturization technology.

Accordingly, the present invention seeks to identify humectants and humectancy systems which moisturize better than the known ones yet do not suffer from their disadvantages.

### SUMMARY OF THE INVENTION

A cosmetic product for moisturizing skin which is delivered through a heating device is provided which includes:
(i) a cosmetic composition having from about 0.01 to about 20% by weight of niacinamide in a cosmetically acceptable carrier;
(ii) a package containing the cosmetic composition; and
(iii) instructions associated with the package describing use of the cosmetic composition which includes delivering the cosmetic composition into the device, applying heat to the composition, thereafter activating a dispensing mechanism of the device and transferring dispensed heated composition to a consumer's skin.

Further, a method for moisturizing skin is provided which includes:
(i) providing a cosmetic composition having from about 0.01 to about 20% by weight of niacinamide in a cosmetically acceptable carrier;
(ii) providing a heating device including a chamber for receiving the cosmetic composition, a heating element for imparting heat to the cosmetic composition, and an outlet for dispensing heated cosmetic composition; and
(iii) dispensing from the heating device and applying the heated cosmetic composition onto the skin.

### DETAILED DESCRIPTION OF THE INVENTION

Now it has been found that niacinamide containing cosmetic compositions when applied at elevated temperatures to the skin result in an improved rate of moisturization. A key component of the cosmetic compositions of this invention is niacinamide. This material is alternatively known as nicotinic acid amide and vitamin B3. Amounts of niacinamide may range from about 0.01 to about 20%, preferably from about 0.1 to about 10%, more preferably from about 0.5 to about 7%, optimally from about 1 to about 5% by weight of the composition.

Dispensing devices for heating cosmetic fluid compositions have-been described in U.S. Pat. No. 6,216,911 B1; U.S. Patent Application Publication 2002/0108965 A1 and U.S. Pat. No. 6,056,160, the specifications of which are herein incorporated by reference. Also there are commercial devices available. One device is sold by Conair Corporation of Stamford, Connecticut under the designation HLD 31 and HLD 20, and another by New Sensations LLC of Englewood, Colo. under the brand New Sensation Lotion Spa.

Heating devices of the present invention are best operated to deliver a composition that exhibits a dispensed temperature between about 30° to about 60° C., more preferably from 38° C. to 54° C., even more preferably from 40° to 49° C. and optimally from 42° to 46° C.

Compositions of the present invention will also include a cosmetically acceptable carrier. Water is the most preferred carrier. Amounts of water may range from about 1 to about 99%, preferably from about 5 to about 90%, more preferably from about 35 to about 70%, optimally between about 40 and about 60% by weight. Ordinarily the compositions will be water and oil emulsions of the W/O or O/W variety. Triplex emulsions which are w/o/w or o/w/o may also be suitable.

Other cosmetically acceptable carriers may include mineral oils, silicone oils, synthetic or natural esters, fatty acids and alcohols and humectants. Amounts of these materials may range from about 0.1 to about 50%, preferably from about 0.1 to about 30%, more preferably from about 1 to about 20% by weight of the composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, preferably from about 4 to about 5, silicon atoms.

Linear volatile silicone materials generally have viscosities less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C. Crosslinked silicone elastomers such as DC9040 and DC9045 can be useful.

Among suitable esters are:
(1) Alkenyl or alkyl esters of fatty acids having 10 to 20 carbon atoms. Examples thereof include isopropyl palmitate, isopropyl isostearate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate.
(5) Sterols esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Adjunct humectants of the polyhydric alcohol-type may also be included in the compositions of this invention. The humectant aids in increasing the effectiveness of the emollient, reduces scaling, stimulates removal of built-up scale and improves skin feel. Typical polyhydric alcohols include glycerol (also known as glycerin), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the adjunct humectant is preferably glycerin. The amount of adjunct humectant may range anywhere from 0.5 to 30%, preferably between 1 and 15% by weight of the composition.

Emulsifiers may be present in cosmetic compositions of the present invention. Total concentration of the emulsifier may range from about 0.1 to about 40%, preferably from about 1 to about 20%, optimally from about 1 to about 5% by weight of the total composition. The emulsifier may be selected from the group consisting of anionic, nonionic, cationic and amphoteric actives. Particularly preferred nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di- C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01 % to about 2% by weight of the composition.

Thickening agents may be included in compositions of the present invention. Particularly useful are the silicates and the polysaccharides. Examples include starches, natural/synthetic gums and cellulosics. Representative of the starches are chemically modified starches such as aluminum starch octenylsuccinate. Suitable gums include xanthan, sclerotium, pectin, karaya, arabic, agar, guar, carrageenan, alginate and combinations thereof. Suitable cellulosics include hydroxypropyl cellulose, hydroxypropyl methylcellu lose, ethylcellu lose and sodium carboxy methylcellu lose. Synthetic polymers are still a further class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature of Sodium Acrylate/Sodium Acryloyldimethyl Taurate and Acryloyl Dimethyltaurate/Vinyl Pyrrolidone Copolymer.

Amounts of the thickener may range from about 0.001 to about 5%, preferably from about 0.1 to about 2%, optimally from about 0.2 to about 0.5% by weight.

Colorants, fragrances and abrasives may also be included in compositions of the present invention. Each of these substances may range from about 0.05 to about 5%, preferably between 0.1 and 3% by weight.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following Examples will more fully illustrate the embodiments of this invention. All parts, percentages and proportions referred to herein and in the appended claims are by weight unless otherwise indicated.

### EXAMPLE 1

A typical body lotion employing niacinamide according to this invention is disclosed in Table I.

TABLE I

| Component | Weight % |
|---|---|
| Glycerin | 10.00 |
| Stearic Acid | 2.34 |
| Magnesium Aluminum Silicate | 1.75 |
| Silicone 50 ct | 1.50 |
| Niacinamide | 1.50 |
| Glyceryl Monostearate/Stearamide AMP | 1.38 |
| Isopropylmyristate | 1.30 |
| Petrolatum | 1.25 |
| Triethanolamine (99%) | 0.70 |
| Glycerol Monostearate | 0.65 |
| Silicone DC 1501 ® | 0.50 |
| Cetyl Alcohol | 0.37 |
| Fragrance | 0.30 |
| DMDM Hydantoin | 0.17 |
| Titanium Dioxide | 0.10 |
| Glydant Plus ® | 0.09 |
| Disodium EDTA | 0.05 |
| Water | Balance |

The lotion of Table 1 is formulated in the following manner. A reactor is charged with the deionized water and disodium EDTA. Heat is applied till 60° C. in combination with stirred mixing. Magnesium aluminum silicate is added to the reactor and the temperature maintained at 77-80° C. for 10 to 15 minutes. In a separate vessel, the oil phase components are added. Light mixing of the batch is performed with heating in a water bath to 75-77° C. The water reactor is maintained at 60-65° C. and slow addition occurred for glycerin, niacinamide, titanium dioxide and triethanolamine. Continuous mixing is done until the aqueous system is uniform. Very slowly the oil phase is added to the water phase at 75-77° C. under moderate mixing. After full emulsification, the batch is agitated for a further 5 minutes. Thereupon the resultant emulsion is homogenized using an ARDE Barenco® apparatus for 20-30 seconds at 35%. The resultant system is then topped with further deionized water. Cooling is then begun with a large sweep (50 rpm) mixer. Preservatives Glydant Pluse® and DMDM Hydantoin is then added with the batch held at 50-55° C. Thereafter the silicone oils are added to the batch. At a temperature of 45-50° C., the fragrance is charged to the reactor. Heating is then discontinued and mixing stopped when the temperature reaches 38-40° C.

The lotion is charged into a Conair HLD 31® Lotion Dispenser. The lotion is heated to a temperature between 38° C. and 54° C. A measured amount of sample is then dispensed from the heated dispenser. This sample amount is placed upon a panelist's forearm. On an opposite forearm of the panelist, a cool (25° C.) identical sample amount of lotion is placed for evaluation. Within 5 minutes of deposition on the forearm, the pre-heated sample will have an improved moisturization. The non-heated sample left on the panelist's other forearm will exhibit a much drier, less moisturized skin.

### EXAMPLE 2

A series of experiments were conducted to evaluate efficacy of the cosmetic composition when applied as a heated substance to skin. More particularly, these experiments evaluated the effectiveness of warm niacinamide to enhance the rate of moisturization of skin.

Tests herein are based on ASTM E-96 which measures water vapor permeability via a cup method. Our method utilized a TAPPI cup custom miniaturized to have a diameter of 1 cm. The procedure involved placing 400 micro-liters of water into a base of the cup. Porcine skin was placed over a mouth of the cup. The experimental cells requiring sample deposit utilized a micropipette delivering 2 mg/cm² of sample onto the skin. We note that this distribution is equivalent to a standard amount of lotion recommended to consumers for placement on their body. The cup was then sealed with an appropriate O-ring and screw cap. Gravimetric weight was taken on an analytical balance prior to placing the cup in an oven at 32° C. for 24 hours. Subsequent to the oven conditioning, the cup was re-weighed to determine loss of water. Calculations were then conducted to state the rate of water loss as mg/cm²/hr. Results are recorded in Table 11 below as the change (delta) in mg/cm²/hr. The more negative the delta value, the better the performance in moisturization. Note that the sample size for statistical purposes was three skin pieces per cell. The surface area for each skin piece was 0.739 cm².

TABLE II

| | | | % |
|---|---|---|---|
| | Delta | Stand. | Improvement |
| | (mg/cm²/hr) | Dev. | Over Control |
| Sample Applied at 45° C. | | | |
| No product | −0.03 | 0.12 | — |
| Formula Without Niacinamide | −2.34 | 0.88 | — |
| Formula with 3% Niacinamide | −4.46 | 0.87 | 90.6 |
| Sample Applied at 21° C. | | | |
| No product | −0.01 | 0.11 | — |
| Formula Without Niacinamide | −1.43 | 0.36 | — |
| Formula with 3% Niacinamide | −1.67 | 0.15 | 16.7 |
| Sample Applied at 21° C. | | | |
| Irradiated 2.5 min. at 45° C. | | | |
| No product | 0.09 | 0.17 | — |
| Formula Without Niacinamide | −1.39 | 0.14 | — |
| Formula with 3% Niacinamide | −1.68 | 0.13 | 21.0 |

In a first set of the experiments it is shown when the base lotion formula (Example 1) was applied without niacinamide at 45° C. to the skin, there was an improvement in moisturization (delta was −2.34) over no lotion applied. Incorporating 3% niacinamide to the base lotion resulted in a delta of −4.46; this is a 90.6% improvement over the control experiment.

In a second series, we evaluated 21° C. (room temperature) as the application temperature. Under these conditions, the niacinamide fortified base lotion had a delta of −1.67 amounting to a 16.7% improvement. The experiments of the second group demonstrate that applying the niacinamide formulated lotion warm has moisturization benefits over a room temperature application.

Finally, a third set of experiments evaluated the benefit of post-application heat treatment. Here the samples were applied at room temperature (21° C.) to the skin. After one minute the area with the lotion was heated by an infrared lamp to obtain 2.5 minutes of 45° C. warmth. The delta value for the 3% niacinamide formulated lotion gave a delta value of −1.68. This is a 21% improvement over the control. However, this improvement was only marginally better than the non-warming result at 21° C. and very substantially inferior to that of the 90.6% improvement where lotion was applied at 45° C. From these experiments we conclude that a niacinamide lotion formula when applied warm to skin has a greater affect than when applied cold. Moreover, warming after application of the cold lotion does not have the same effect as applying a warm lotion immediately onto skin.

What is claimed is:

1. A cosmetic product for moisturizing skin which is delivered through a heating device comprising:
(i) a cosmetic composition having from about 0.01 to about 20% by weight of niacinamide in a cosmetically acceptable carrier;
(ii) a package containing the cosmetic composition; and
(iii) instructions associated with the package describing use of the cosmetic composition which includes delivering the cosmetic composition into the device, applying heat to the composition, thereafter activating a dispensing mechanism of the device and transferring dispensed heated composition to a consumer's skin.

2. The product according to claim 1 wherein the heated composition is at a temperature from 38° to 54° C.

3. The product according to claim 1 wherein the heated composition is at a temperature from 40° to 49° C.

4. The product according to claim 1 wherein niacinamide is present in an amount from about 0.5 to about 7% by weight of the composition.

5. The product according to claim 1 wherein niacinamide is present in an amount from about 1 to about 5% by weight of the composition.

6. A method for moisturizing skin comprising:
(i) providing a cosmetic composition comprising from about 0.01 to about 20% by weight of niacinamide in a cosmetically acceptable carrier;
(ii) providing a heating device comprising a chamber for receiving the cosmetic composition, a heating element for imparting heat to the cosmetic composition, and an outlet for dispensing heated cosmetic composition; and
(iii) dispensing from the heating device and applying the heated cosmetic composition onto the skin.

7. The method according to claim 6 wherein the heated cosmetic composition is at a temperature from 38° to 54° C.

8. The method according to claim 6 wherein the heated composition is at a temperature from 40° to 49° C.

9. The method according to claim 6 wherein niacinamide is present in an amount from about 0.5 to about 7% by weight of the composition.

10. The method according to claim 6 wherein niacinamide is present in an amount from about 1 to about 5% by weight of the composition.
